SEQUENCE LISTING (6 sequences)

SEQ ID NO: 1 - protein, 49 aa, human
NPWVCDCRARPLWAWLQKFRGSSSEVPCSLPQRLAGRDLKRLAANDLQG

SEQ ID NO: 2 - protein, 49 aa, mouse
NPWVCDCRARPLWAWLQKFRGSSSEVPCNLPQRLADRDLKRLAASDLEG

SEQ ID NO: 3 - protein, 49 aa, bovine
NPWVCDCRARPLWAWLQKFRGSSSGVPSNLPQRLAGRDLKRLATSDLEG

SEQ ID NO: 4 - protein, 47 aa, Artificial Sequence
  Description of Artificial Sequence Synthetic peptide sequence
PALCLCRARPLWAWLQKFRGSSSEVPCSLPQRLAGRDLKRLAANDLA

SEQ ID NO: 5 - protein, 40 aa, Artificial Sequence
  Description of Artificial Sequence Synthetic peptide sequence
RPLWAWLQKFRGSASEVPCSLPQRLAGRDLKRLAANDLQG

SEQ ID NO: 6 - protein, 43 aa, Artificial Sequence
  Description of Artificial Sequence Synthetic peptide sequence
QPAVLDCRARPLWAWLQKFRGSSSEVPLSLPQRLAGRDLKRLA

(19) United States
(12) Patent Application Publication
(10) Pub. No.: US 2003/0113325 A1
(43) Pub. Date: Jun. 19, 2003

(54) Reducing myelin-mediated inhibition of axon regeneration

(76) Inventors: Zhigang He, Boston, MA (US); Kevin C. Wang, Boston, MA (US); Vuk Koprivica, Boston, MA (US); Jieun A. Kim, Boston, MA (US)

Correspondence Address: RICHARD ARON OSMAN, SCIENCE AND TECHNOLOGY LAW GROUP, 75 DENISE DRIVE, HILLSBOROUGH, CA, 94010

(21) Appl. No.: 10/006,002

(22) Filed: Dec. 3, 2001

(51) Int. Cl. A61K 39/395; G01N 33/53; G01N 33/567

(52) U.S. Cl. 424/146.1; 435/7.2

(57) ABSTRACT

Oligodendrocyte-myelin glycoprotein (OMgp)-specific binding agents are used to reduce OMgp-mediated axon growth inhibition. Mixtures of axons and OMgp and mixtures of Nogo receptor (NgR) and OMgp are used in pharmaceutical screens to characterize agents as inhibiting binding of NgR to OMgp and promoting axon regeneration.

This work was supported by Federal Grant No. 1R21NS41999-01 from NINDS. The government may have rights in any patent issuing on this application.

### INTRODUCTION

1. Field of the Invention

The invention is in the field of reducing meylin-mediated inhibition of axon regeneration.

2. Background of the Invention

Most axons in the adult mammalian central nervous system (CNS) have little innate ability for repair after injury (Horner and Gage, 2000). Examination of post-lesioned axons in the adult nervous system reveals that their proximal ends are able to form growth cones, the primary navigating entity of growing axons, that appear both morphologically and functionally identical to those of developing nerve fibers (reviewed by Tessier-Lavgine and Goodman, 2000). Studies over the past two decades have identified a number of guidance cues that can influence the motility and directionality of projecting axons during embryonic development (Tessier-Lavigne and Goodman, 1996; Song and Poo, 2001). It is believed that the combined influences of attractants and repellents orchestrate the precise motile behavior of individual axons. Conversely, either a lack of permissive cues and/or the presence of dominant inhibitors in the adult CNS seem to contribute significantly to the inability of lesioned axons to regenerate (Schwab & Bartholdi, 1996; Fournier and Strittmatter, 2001).

In addition to the actual physical barrier presented by glial scarring at the lesion sites (KcKeon et al., 1991; Davies et al., 1997; Moon et al., 2001), inhibitory factors in oligodendrocyte-derived myelin clearly play a role in limiting axon regeneration. Immobilized CNS myelin proteins have been shown to potently inhibit axon outgrowth from a variety of neurons in vitro (Schwab and Caroni, 1988; Savio and Schwab, 1989). In addition, anti-myelin antibodies have been used to neutralize the inhibitory effects of myelin and, more importantly, stimulate regeneration of the corticospinal tract in vivo (Schnell and Schwab, 1990; Bregmann et al., 1995; Huang et al., 1999). Most of the efforts towards identifying these myelin-associated inhibitors thus far have centered on assaying biochemical fractions of CNS myelin for growth-inhibitory activity in vitro and then isolating the corresponding molecules (Caroni and Schwab, 1988; McKerracher et al., 1994; Spillmann et al., 1998; Niederost et al., 1999). Several myelin components have been identified as putative inhibitors of regeneration through such approaches. One such component is myelin associated glycoprotein (MAG), a transmembrane protein with a five immunoglobulin domain-harboring extracellular region (Arquint et al., 1987; Salzer et al., 1987). Even though MAG is capable of inhibiting axon outgrowth from different types of cultured neurons (McKerracher et al., 1994; Mukhopadhyay et al., 1994; Li et al., 1996; Tang et al., 1997), knockout animals provide conflicting data on the effects of removing the MAG protein product on axon regeneration in vivo (Bartsch et al., 1995; Li et al., 1996; Schafer et al., 1996). In addition to MAG, neurite outgrowth-inhibitory activity has also been found to associate with chondroitin sulfate proteoglycans (CSPGs) in CNS myelin (Niederost et al., 1999). However, it is unclear whether this inhibitory activity results from CSPGs themselves or from a combination with additional factors.

In addition to MAG and CSPGs, another putative inhibitor, Nogo/NI-250 (Caroni and Schwab, 1988), has attracted much attention because an anti-NI-250 monoclonal antibody, IN-1, had been shown to neutralize the growth-inhibitory effects of myelin-associated inhibitors both in vitro and in vivo. Remarkably, IN-1 treatment resulted in long-distance fiber growth and increased axonal sprouting within the adult CNS (Schnell and Schwab, 1990; Bregmann et al., 1995; Thallmair et al., 1998). The partial-peptide sequence of biochemically purified NI-250 (Spillmann et al., 2000) allowed several groups to clone the corresponding cDNA which encodes three Nogo isoforms, designated Nogo-A (presumably NI-250), -B, and -C, presumably generated from alternative splicing or differential promoter usage (Chen et al., 2000; GrandPre et al., 2000; Prinjha et al., 2000). Surprisingly, Nogo-A, or Reticulon 4-A, appears to be a member of the reticulon protein family, and associates primarily with the endoplasmic reticulum (ER) (GrandPre et al., 2000). Nogo-A protein is believed to contain at least two transmembrane domains. Interestingly, both the amino-terminal cytoplasmic (amino-Nogo) (Chen et al., 2000; Prinjha et al., 2000; Fournier et al., 2001) and the lumenal/extracellular (Nogo-66) (GrandPre et al., 2000; Fournier et al., 2001) domains of Nogo are able to inhibit axon growth in vitro. Insights into the signaling mechanism(s) that mediate the inhibitory activity of Nogo came with the recent identification of a functional receptor for Nogo-66 by expression cloning (Fournier et al., 2001). The Nogo-66 receptor (NgR), a protein which associates with the plasma membrane through a glycosylphosphatidylinositol (GPI) anchor, is expressed in most postnatal neuronal populations and binds Nogo-66 with high affinity (Fournier et al., 2001). Furthermore, early embryonic chick retinal ganglion cells that are normally insensitive to Nogo-66 become responsive upon expression of NgR (Fournier et al., 2001), suggesting that NgR is able to mediate the activity of Nogo-66. However, it is unclear how the primarily intracellularly localized Nogo protein reaches and acts on regenerating axons.

Previous biochemical studies have demonstrated the presence of additional inhibitory activities of unknown molecular identity (McKerracher et al., 1994; Spillmann et al., 1999; Niederost et al., 1999). As most myelin proteins are assumed to be associated with the plasma membrane of axon-ensheathing oligodendrocytes, we reasoned that these proteins could either be in the transmembrane form or be tethered to the membrane with covalent linkers. As both MAG and Nogo are transmembrane proteins, and the glycosylphosphatidylinositol (GPI)-mediated covalent linkage appears to be the most common structural feature of many membrane-associated axon guidance molecules (for example, Ranscht and Dours-Zimmermann, 1991; Xu et al., 1998; Nakashiba et al., 1999; O'Leary and Wilkinson, 1999), we decided to investigate the possibility that GPI-linked CNS myelin proteins may play a role in inhibiting axon regeneration. By utilizing phosphatidylinositol-specific phospholipase C (PI-PLC) to release GPI-linked proteins from CNS myelin, we found these proteins to have a potent growth cone-collapsing activity. We found that oligodendrocyte-myelin glycoprotein (OMgp), a previously identified GPI-linked CNS myelin protein with unknown function (Mikol and Stefansson, 1988; Habib et al., 1998a; Habib et al. 1998b), provides such inhibitory activity. Furthermore, through the use of both loss- and gain-of-function experiments, we demonstrate that OMgp acts through NgR to inhibit axon regeneration.

### SUMMARY OF THE INVENTION

The invention provides methods and compositions for reducing OMgp-mediated axon growth inhibition. In one embodiment, the method comprising steps (a) contacting a mixture comprising an axon and isolated OMgp with an agent and under conditions wherein but for the presence of the agent, the axon is subject to growth inhibition mediated by the OMgp; and (b) detecting resultant reduced axon growth inhibition. In an alternative embodiment, the method comprises steps: (a) contacting a mixture comprising an axon and OMgp with an exogenous OMgp-specific binding agent and under conditions wherein but for the presence of the agent, the axon is subject to growth inhibition mediated by the OMgp, whereby the agent binds the OMgp and reduces the growth inhibition; and (b) detecting resultant reduced axon growth inhibition.

These methods may be practiced with isolated neurons in vitro, or with neurons in situ. Suitable agents include (i) a candidate agent not previously characterized to bind OMgp nor reduce axon growth inhibition mediated by OMgp; (ii) a candidate agent not previously characterized to reduce axon growth inhibition mediated by OMgp; (iii) an OMgp-specific antibody fragment; (iv) a soluble NgR peptide sufficient to specifically bind the OMgp and competitively inhibit binding of the OMgp to NgR; etc. In more particular embodiments, the recited isolated OMgp consists essentially of OMgp, particularly wherein the OMgp is soluble and GPI-cleaved and/or the OMgp is recombinantly expressed on a surface of a cell.

The invention also provides methods and compositions for characterizing an agent as inhibiting binding of NgR to OMgp. In one embodiment, this method comprising the steps (a) incubating a mixture comprising NgR, OMgp and an agent under conditions whereby but for the presence of the agent, the NgR and OMgp exhibit a control binding; and (b) detecting a reduced binding of the NgR to the OMgp, indicating that the agent inhibits binding of the NgR to the OMgp.

The method may be practiced in a variety of alternative embodiments, such as (i) wherein at least one of the NgR and OMgp is soluble and GPI-cleaved; (ii) wherein one of the NgR and OMgp is soluble and GPI-cleaved and the other is membrane-bound; (iii) wherein at least one of the NgR and OMgp is recombinantly expressed on a surface of a cell; etc.

The invention also provides compositions and mixtures specifically tailored for practicing the subject methods. For example, an in vitro mixture for use in the subject binding assays comprises NgR, OMgp and an agent, wherein at least one of the NgR and OMgp is soluble and GPI-cleaved. Kits for practicing the disclosed methods may also comprise printed or electronic instructions describing the applicable subject method.

### DESCRIPTION OF PARTICULAR EMBODIMENTS OF THE INVENTION

The following descriptions of particular embodiments and examples are offered by way of illustration and not by way of limitation. Unless contraindicated or noted otherwise, in these descriptions and throughout this specification, the terms “a” and “an” mean one or more, the term “or” means and/or and polynucleotide sequences are understood to encompass opposite strands as well as alternative backbones described herein.

In one embodiment, the invention provides a method for reducing axon growth inhibition mediated by OMgp and detecting resultant reduced axon growth inhibition, the method comprising steps: contacting a mixture comprising an axon and isolated OMgp with an agent and under conditions wherein but for the presence of the agent, the axon is subject to growth inhibition mediated by the OMgp; and detecting resultant reduced axon growth inhibition, indicating that the agent reduces axon growth inhibition mediated by OMgp.

The recited axons are mammalian neuron axons, preferably adult neural axons, which may be peripheral or, preferably CNS neuron axons. As exemplified below, the method may be applied to neural axons in vitro or in situ.

OMgp is a natural, mammalian CNS myelin glycoprotein (see, Habib et al. 1998a, 1998b) which functions as a ligand of the Nogo Receptor (NgR) on CNS axons. OMgp cDNA has been cloned from several species, including human (Genbank Accn No. NM_{—}002544), mouse (Genbank Accn No. NM_{—}019409), and cow (Genbank Accn No. S45673). Note that OMgp cDNA encodes two alternative initiating methionine residues; compare, Genbank Accession Nos. M63623 (human) and S67043 (mouse). OMgp may be membrane-bound through a GPI linkage or cleaved therefrom. As exemplified herein, OMgp may be obtained on or cleaved from naturally expressing myelin. Also as exemplified herein, OMgp may also be expressed recombinantly in suitable recombinant expression systems, wherein functional expression may be confirmed by the growth cone collapsing assays described herein.

The recited isolated OMgp is provided isolated from other components of OMgp's natural myelin mileau, which may be effected by purification from such components or expression of the OMgp in a non-natural system. In particular embodiments, the isolated OMgp is accompanied by other components which provide or interfere with or alter the axon growth inhibitory or NgR binding activity of the OMgp. Preferred isolated OMgp is purified or recombinantly expressed, particularly on a surface of a cell.

The recited agent may be characterized as an OMgp-specific binding agent or, particularly as applied to pharmaceutical screens, an agent not previously characterized to bind OMgp nor reduce axon growth inhibition mediated by OMgp, wherein the agent is a candidate agent and the detecting step characterizes the candidate agent as reducing axon growth inhibition mediated by OMgp. Similarly, the agent may be a candidate agent not previously characterized to reduce axon growth inhibition mediated by OMgp, wherein the detecting step characterizes the candidate agent as reducing axon growth inhibition mediated by OMgp.

Detailed protocols for implementing the recited steps are exemplified below and/or otherwise known in the art as guided by the present disclosure. The recited contacting and detecting steps are tailored to the selected system. In vitro systems provide ready access to the recited mixture using routine laboratory methods, whereas in vivo systems, such as intact organisms or regions thereof, typically require surgical or pharmacological methods. More detailed such protocols are described below. Similarly, the detecting step is effected by evaluating different metrics, depending on the selected system. For in vitro binding assays, these include conventional solid-phase labeled protein binding assays, such as ELISA-type formats, solution-phase binding assays, such as fluorescent polarization or NMR-based assays, etc. For cell-based or in situ assays, metrics typically involve assays of axon growth as evaluated by linear measure, density, host mobility or other function improvement, etc.

In another embodiment, the invention provides a method for reducing axon growth inhibition mediated by OMgp and detecting resultant reduced axon growth inhibition by (a) contacting a mixture comprising an axon and OMgp with an exogenous OMgp-specific binding agent and under conditions wherein the agent binds the OMgp and but for the presence of the agent, the axon is subject to growth inhibition mediated by the OMgp, and (b) detecting resultant reduced axon growth inhibition.

This protocol may similarly be practiced with in vitro or in vivo, particularly in situ, mixtures. Note that in this embodiment, the agent is necessarily an exogenous OMgp-specific binding agent and the recited OMgp need not be isolated, i.e. it may be present in the context of its native myelin. Accordingly, this aspect of the invention provides methods for reducing axon growth inhibition mediated by OMgp in its native mileau. By reducing axon growth inhibition, the methods assist the repair of axons following injury or trauma, such as spinal cord injury. In addition, the methods may be applied to alleviate dysfunction of the nervous system due to hypertrophy of neurons or their axonal projections, such as occurs in diabetic neuropathy.

An OMgp-specific binding agent exogenous to an axon or mixture comprising an axon is not naturally present with the axon or mixture. The OMgp-specific binding agents specifically bind the OMgp of the recited mixture and thereby functionally inhibit the axon collapse and/or NgR binding mediated by the OMgp. We have exemplified suitable OMgp binding agents from diverse structures. Initial agents were identified by selecting high affinity OMgp binders from natural NgR peptides. These assays identified a number of OMgp-specific NgR peptides encompassing NgR LLR sequences, including the exemplified species: hNR260/308, mNR260/308 and rNR260/308. Natural OMgp-specific NgR peptide sequences were subject to directed combinatorial mutation and binding analysis. Resultant synthetic-sequence OMgp-specific peptides include the exemplified species: s1NGR260/308, s2NR260/308 and s3NR260/308. We also used a variety of OMgp peptide immunogens to generate OMgp-specific antibodies and antibody fragments, including the exemplified monoclonal antibodies OM-H2276 and OM-H5831 and the exemplified fragments OMF-H7712 and OMF-H6290. OMgp-specific binding agents are also found in compound libraries, including the exemplified commercial fungal extract and a synthetic combinatorial organo-pharmacophore-biased libraries. Structural characterization of the exemplified OMgp binding agents (XR-178892, XR-397344, XR-573632, SY-73273M, SY-32340L and SY-95734E) is effected by conventional organic analysis.

Of particular interest are size-minimized NgR LLR peptides which effectively compete for OMgp ligand binding. We synthesized and screened large libraries of NgR peptides for their ability to bind OMgp and thereby reduce OMgp-mediated axon growth inhibition. This work identified numerous competitive binding peptides of varying length within a 49 amino acid region of a NgR C-terminal leucine rich repeat, exemplified with human, mouse and rat repeat sequences (hNR260/308, SEQ ID NO:1; mNR260/308, SEQ ID NO:2; and rNR260/308, SEQ ID NO: 3). Competitive peptides demonstrating >20% competitive activity compared with the source 49 mer are subject to combinatorial mutagenesis to generate synthetic peptide libraries from which we screen for even higher affinity binders. Preferred competitive peptides consist, or consist essentially of a size-minimized sequence within the disclosed human source 49 mer, preferably a sequence of fewer than 48, 38, 28 or 18 residues, wherein at least 6, 8, 12 or 16 residues are usually required for specific binding. Obtaining additional such native sequence and synthetic competitive peptides involves only routine peptide synthesis and screening in the disclosed binding and growth assays.

In particular applications, the target cells are injured mammalian neurons in situ, e.g. Schulz M K, et al., Exp Neurol. February 1998; 149(2): 390-397; Guest J D, et al., J Neurosci Res. Dec. 1, 1997; 50(5): 888-905; Schwab M E, et al., Spinal Cord. July 1997; 35(7): 469-473; Tatagiba M, et al., Neurosurg March 1997; 40(3): 541-546; and Examples, below. For these in situ applications, compositions comprising the OMgp binding agent may be administered by any effective route compatible with therapeutic activity of the compositions and patient tolerance. For example, for CNS administration, a variety of techniques is available for promoting transfer of therapeutic agents across the blood brain barrier including disruption by surgery or injection, drugs which transiently open adhesion contact between CNS vasculature endothelial cells, and compounds which facilitate translocation through such cells. The compositions may also be amenable to direct injection or infusion, intraocular administration, or within/on implants e.g. fibers such as collagen fibers, in osmotic pumps, grafts comprising appropriately transformed cells, etc.

In a particular embodiment, the binding agent is delivered locally and its distribution is restricted. For example, a particular method of administration involves coating, embedding or derivatizing fibers, such as collagen fibers, protein polymers, etc. with therapeutic agents, see also Otto et al. (1989) J Neurosci Res. 22, 83-91 and Otto and Unsicker (1990) J Neurosc 10, 1912-1921. The amount of binding agent administered depends on the agent, formulation, route of administration, etc. and is generally empirically determined and variations will necessarily occur depending on the target, the host, and the route of administration, etc.

The compositions may be advantageously used in conjunction with other neurogenic agents, neurotrophic factors, growth factors, anti-inflammatories, antibiotics etc.; and mixtures thereof, see e.g. Goodman & Gilman's The Pharmacological Basis of Therapeutics, 9^{th} Ed., 1996, McGraw-Hill. Exemplary such other therapeutic agents include neuroactive agents such as in Table 1.

TABLE 1
Neuroactive agents which may be used in conjunction with OMgp
binding agents.

| | | |
|---|---|---|
| NGF | Heregulin | Laminin |
| NT3 | IL-3 | Vitronectin |
| BDNF | IL-6 | Thrombospondin |
| NT4/5 | IL-7 | Merosin |
| CNTF | Neuregulin | Tenascin |
| GDNF | EGF | Fibronectin |
| HGF | TGFa | F-spondin |
| bFGF | TGFb1 | Netrin-1 |
| LIF | TGFb2 | Netrin-2 |
| IGF-I | PDGF BB | Semaphorin-III |
| IGH-II | PDGF AA | L1-Fc |
| Neurturin | BMP2 | NCAM-Fc |
| Percephin | BMP7/OP1 | KAL-1 |

Abbreviations:
NGF, nerve growth factor;
NT, neurotrophin;
BDNF, brain-derived neurotrophic factor;
CNTF, ciliary neurotrophic factor;
GDNF, glial-derived neurotrophic factor;
HGF, hepatocyte growth factor;
FGF, fibroblast growth factor;
LIF, leukemia inhibitory factor;
IGF, insulin-like growth factor;
IL, interleukin;
EGF, epidermal growth factor;
TGF, transforming growth factor;
PDGF, platelet-derived growth factor;
BMP, bone morphogenic protein;
NCAM, neural cell adhesion molecule.

In particular embodiments, the OMgp binding agent is administered in combination with a pharmaceutically acceptable excipient such as sterile saline or other medium, gelatin, an oil, etc. to form pharmaceutically acceptable compositions. The compositions and/or compounds may be administered alone or in combination with any convenient carrier, diluent, etc. and such administration may be provided in single or multiple dosages. Useful carriers include solid, semi-solid or liquid media including water and non-toxic organic solvents. As such the compositions, in pharmaceutically acceptable dosage units or in bulk, may be incorporated into a wide variety of containers, which may be appropriately labeled with a disclosed use application. Dosage units may be included in a variety of containers including capsules, pills, etc.

The invention also provides pharmaceutical screens for inhibitors of OMgp-NgR binding, particularly, methods for characterizing an agent as inhibiting binding of NgR to OMgp by: (a) incubating a mixture comprising NgR, OMgp and an agent under conditions whereby but for the presence of the agent, the NgR and OMgp exhibit a control binding; and (b) detecting a reduced binding of the NgR to the OMgp, indicating that the agent inhibits binding of the NgR to the OMgp.

NgR is a natural, mammalian neural axon protein (Fournier et al., 2001, Nature 409, 341-46) which functions as a receptor for Nogo66 and for OMgp. NgR cDNA has been cloned from several species, including human (Genbank Accn No. BC011787), mouse (Genbank Accn No. NM-022982), and rat (Genbank Accn No. AY028438). NgR may be membrane-bound through a GPI linkage or cleaved therefrom. As exemplified herein, NgR may be obtained on or cleaved from naturally expressing myelin. Also as exemplified herein, NgR may also be expressed recombinantly in suitable recombinant expression systems, wherein functional expression may be confirmed by the growth cone collapsing assays described herein.

The screening method is amenable to a wide variety of different protocols. For example, in particular embodiments, at least one of the NgR and OMgp is soluble and GPI-cleaved, one of the NgR and OMgp is soluble and GPI-cleaved and the other is membrane-bound, and at least one of the NgR and OMgp is recombinantly expressed on a surface of a cell.

The invention also provides compositions and mixtures specifically tailored for practicing the subject methods. For example, an in vitro mixture for use in the subject binding assays comprises premeasured, discrete and contained amounts of NgR, OMgp and an agent, wherein at least one of the NgR and OMgp is soluble and GPI-cleaved. Kits for practicing the disclosed methods may also comprises printed or electronic instructions describing the applicable subject method.

### EXAMPLES

Identification of OMgp as an inhibitor of axon outgrowth. To examine whether any GPI-linked proteins in CNS myelin may act as inhibitors of axon regeneration, we treated purified bovine white matter myelin with phosphatidylinositol-specific phospholipase C (PI-PLC). PI-PLC cleaves GPI anchors at the junction between the bilayer-associated diacylglycerol and the peptide-associated phosphoinositol ring, resulting in the release of the polypeptide chain with its attached anchor glycan from the lipid bilayer (Low, 1987). The PI-PLC-released proteins were then examined for their ability to alter growth cone morphology in a growth cone collapse assay using embryonic day 13 (E13) chick dorsal root ganglia (DRG)(Luo et al., 1993; He and Tessier-Lavigne, 1997; Fournier et al., 2001). Our data show that PI-PLC-released CNS myelin proteins, when added to the DRG culture medium, exhibited potent growth cone collapsing activity.

To further characterize the inhibitory activity in the PI-PLC-released proteins, we analyzed solubilized proteins by SDS-PAGE and silver staining and found that a band of approximately 110 kDa in size was significantly enriched in this fraction. Previous studies have identified several GPI-linked proteins in CNS myelin, including the 120 kDa N-CAM120 (Bhat and Silberberg, 1986), the 62-70 kDa 5′-nucleotidase (Cammer et al., 1985; Zimmermann et al., 1992), the 135 kDa F-3 (Koch et al., 1997), 90 and 120 kDa brevican (Seidenbecher et al., 1995), and the 110 kDa oligodendrocyte-myelin glycoprotein (OMgp, Mikol and Stefansson et al., 1988). Since OMgp was the closest in size to our 110-kDa band, we used anti-OMgp antibodies to detect the enrichment of cleaved OMgp in the PI-PLC supernatants by Western blot. Our data show that anti-OMgp antibodies detected a band of similar size in the PI-PLC-treated supernatants, indicating that OMgp is one of the components released from CNS myelin by PI-PLC.

Next, we examined whether purified recombinant OMgp protein was able to influence the axon outgrowth behavior of cultured neurons. We engineered a construct that drives the expression of a polyhistidine-tagged mouse OMgp protein in COS-7 cells so that the expressed OMgp-His proteins could be readily purified by a nickle-agarose column. Similar to the PI-PLC-treated myelin supernatants, purified OMgp-His protein, but not proteins purified from control vector-transfected COS-7 cells, induced the collapse of growth cones derived from E13 chick DRG neurons. To further demonstrate that the OMgp protein acts as an inhibitor of axon regeneration, we assessed its ability to affect axon outgrowth in cultured neurons. Our data show that OMgp-His inhibited axon outgrowth of cerebellar granule neurons from postnatal day 7-9 (P7-9) rats when the protein was provided in either immobilized or soluble form. The OMgp-induced inhibitory responses were strikingly reminiscent of that brought about by treatment of the neurons with an alkaline phosphatase-fusion protein containing the 66 amino acid lamenal/extracellular domain of Nogo-A (AP-66). Comparable effects were also observed in neurite outgrowth assays with differentiated PC12 cells. Together, these results indicate that OMgp is a novel inhibitor of axon regeneration.

Expression cloning of the Nogo-66 receptor as an OMgp-binding protein. To further investigate the mechanisms by which OMgp inhibits axon outgrowth, we utilized an expression cloning strategy to isolate OMgp-binding proteins. The coding region of OMgp was fused to that of alkaline phosphatase (AP), a readily detectable histochemical reporter (Flanagan and Leder, 1990; Flanagan and Cheng, 2000), and of a polyhistidine tag for expression and subsequent purification of the chimeric protein in COS-7 cells. The purified protein appeared as a major band of about 180 kDa as detected by Western blotting, consistent with the combined sizes of OMgp and AP; a few smaller peptides, apparently degradation products, were also detected in the preparation. The AP-OMgp protein, but not AP alone, bound to axons when applied to the culture medium of P9 granule cells.

We next took advantage of AP-OMgp binding to identify cell surface OMgp-binding proteins by expression cloning (He & Tessier-Lavigne, 1997; Flanagan and Cheng, 2000; Fournier et al., 2001). Pools of a complementary DNA expression library from adult human brain, representing approximately 250,000 independent clones, were transfected into COS-7 cells and screened for the presence of cells that bound AP-OMgp. As expected, un-transfected COS-7 cells did not bind AP-OMgp, but transfection with two pools of 5,000 clones each resulted in a few OMgp-binding cells. After screening several rounds of subpools, we isolated two cDNAs that encoded the OMgp-binding proteins. Sequencing analysis revealed that both cDNAs contained the full-length coding region of the nogo-66 receptor (NgR), which had been previously identified as a high-affinity receptor for the lamenal/extracellular domain of Nogo (Fournier et al., 2001). Upon transfection of the NgR cDNA into neuroblastoma N2A cells, we were able to determine the binding affinity of expressed NgR for OMgp as 5 nM, similar to what had been determined for Nogo-66 (7 nM, Fournier et al., 2001). These data indicate that NgR is a high-affinity OMgp-binding protein. We next performed coimmunoprecipitation experiments by incubating GST or a GST-NgR fusion protein containing the entire extracellular domain of NgR (GST-NgR) with AP or AP-OMgp. Our data show that GST-NgR, but not control GST protein, bound selectively to AP-OMgp, indicating a direct interaction between OMgp and NgR.

OMgp binds the Nogo-66 receptor through its leucine-rich repeat domain. Amino acid sequence analysis indicated that OMgp, like NgR (Fournier et al., 2001), is a GPI-linked protein containing a leucine-rich-repeat (LRR) domain, which has been implicated in mediating different protein-protein interactions (Kobe and Deisenhofer, 1994). In addition, OMgp was predicted to have a C-terminal domain with serine-threonine repeats (Mikol et al., 1990). To determine which region(s) of OMgp is responsible for its binding to the NgR, we generated two additional constructs fusing AP to the N- or C-terminal region of OMgp and used the conditioned medium from COS-7 cells transfected with each of these constructs to test for NgR binding. When comparable amounts of each AP fusion protein were incubated with either control or NgR-expressing CHO cells, AP-OMgp-LRR showed strong binding to NgR-expressing cells, indicating that the LRR domain of OMgp might mediate the interaction between OMgp and NgR. It remains to be determined whether this interaction is mediated by the LRR domains of both proteins or whether OMgp binding to NgR occurs independent of the NgR LRR domain. We also observed weak binding of the AP fusion protein containing only the serine-threonine repeats of OMgp (AP-OM-S/T) to NgR expressing cells. As this domain of OMgp has been proposed to harbor the attachment sites of O-linked carbohydrates (Mikol et al., 1990), we repeated the binding assays in the presence of heparin, a non-specific binding competitor, and found that the AP-OM-S/T and NgR interaction was not affected.

PI-PLC treatment abolishes neuronal responses to OMgp. To assess whether NgR mediates the axon outgrowth-inhibitory effects of OMgp, we took advantage of the fact that the GPI-linked NgR protein can be released by PI-PLC and examined whether PI-PLC treatment could affect the axon responsiveness to OMgp. Consistent with a previous study (Fournier et al., 2001), PI-PLC treatment did not alter the growth cone morphology of E13 chick DRG neurons, but rendered these axons insensitive to Nogo-66. Similarly, PI-PLC treatment also abolished the growth cone-collapse activity of OMgp. However, the growth cone collapse activity of Semaphorin 3A (Sema 3A), known to be mediated by transmembrane receptor molecules including neuropilin-1 and members of the plexin family (reviewed by Raper et al., 2000), was not affected by PI-PLC treatment.

Nogo-receptor confers neuronal responsiveness to OMgp. To assess whether NgR is capable of mediating OMgp-induced inhibitory activity on axon outgrowth, we took a gain-of-function approach to examine whether expression of NgR was able to confer OMgp-responsiveness to otherwise insensitive neurons. It has been shown previously that chick E7 retinal ganglion neurons are insensitive to Nogo-66, but that expression of NgR in these neurons rendered their growth cones to be responsive to Nogo-66 (Fournier et al., 2001). Using the same strategy, we made a recombinant herpes simplex virus (HSV) that drives expression of a flag-tagged full-length NgR in infected neurons. Upon infection, most of the E7 retinal ganglion axons expressed the NgR protein as assessed by immunostaining with an anti-flag antibody. No significant morphological alterations were observed in the HSV-infected neurons. Consistent with a previous study (Fournier et al., 2001), expression of flag-NgR conferred a Nogo-66-induced growth cone collapse response to E7 retinal ganglion cells. Furthermore, the growth cones of NgR-expressing axons also became collapsible by OMgp. In contrast, a control virus driving the expression of LacZ did not alter the axonal responses of the same neurons to either Nogo-66 or OMgp.

To further substantiate the ability of NgR to mediate OMgp-elicited neuronal responses, we screened a number of neuronal cell lines and found that mouse neuroblastoma cells (N2A) are insensitive to both OMgp and Nogo-66. These cells consistently failed to bind the AP-OMgp protein. In order to extend our findings, we established a N2A cell line that stably expresses flag-NgR, and found that neurite outgrowth in these cells was dramatically inhibited when challenged with both soluble and immobilized OMgp-His. The same observations were made in parallel with soluble and immobilized AP-66. Taken together, these results indicate that NgR mediates the axon outgrowth inhibitory activity of OMgp.

Purification and PI-PLC treatment of myelin. Myelin was prepared from white matter of bovine brain according to established protocols (Norton and Poduslo, 1973). In brief, white matter tissues were homogenized in 0.32 M sucrose in phosphate-buffered saline (PBS) and the crude myelin that banded at the interphase of a discontinuous sucrose gradient (0.32M/0.85 M) was collected and purified by two rounds of osmotic shock with distilled water and re-isolation over the sucrose gradient. For PI-PLC treatment, aliquots of myelin suspension in water (10 mg/ml) were incubated with or without 2.5 U/ml PI-PLC (Sigma) at 37° C. for 2 hr, prior to centrifugation (360,000 g for 60 min). The supernatants were concentrated, partitioned in Triton X-114, and used for assays and detection with Western analysis.

Expression cloning and binding experiments. Sequences encoding mouse OMgp were amplified from Marathon-ready mouse cDNA (Clontech) and confirmed by sequencing analysis, prior to subcloning into the expression vector AP-5 (Flanagan and Cheng, 2000) for expressing an AP-OMgp fusion protein tagged with both a polyhistidine and a myc epitope. The resultant plasmid DNA was transfected into COS-7 cells and the secreted protein purified using nickel-agarose resins (Qiagen).

Cell surface binding and expression cloning were performed as described previously (He and Tessier-Lavigne, 1997). To detect AP-OMgp binding, cultures were washed with binding buffer (Hanks balanced salt solution containing 20 mM Hepes, pH 7.5, and 1 mg/ml bovine serum albumin (BSA)). The plates were then incubated with AP-OMgp-containing binding buffer for 75 min at room temperature. After extensive washing and heat inactivation, bound AP fusion proteins were detected by AP staining using NBT and BCIP as substrate. For saturation analysis, we disrupted cells and detected bound AP fusion proteins using r-nitrophenyl phosphate as substrate.

For expression cloning of OMgp-binding proteins, pools of 5,000 arrayed clones from a human brain cDNA library (Origene Technologies, Rockville, Md.) were transfected into COS-7 cells, and AP-OMgp binding was assessed. We isolated single NgR cDNA clones by sub-dividing the pools and sequencing analysis.

Generation of recombinant proteins and virus, immunoprecipitation, and Western analysis. To express recombinant OMgp for function assays, we subcloned the coding region sequence of mouse OMgp (amino acids 23-392) into pSecTag B to express his-tagged OMgp protein (OM-His) in COS-7 cells. The expressed OMgp-His protein was purified using a nickel resin. To construct recombinant herpes simplex viruses (HSV), cDNAs for flag-tagged NgR or LacZ were inserted into the HSV amplicon HSV-PrpUC and packaged into the virus using the helper 5dl1.2, as described previously (Neve et al., 1997). The resultant viruses were purified on a sucrose gradient, pelleted, and resuspended in 10% sucrose. The titer of the viral stocks was ˜4.0×10⁷ infectious units/ml. For each study, aliquots from the same batches of the viral vectors were used. In order to produce recombinant Nogo-66 protein, the sequence of Nogo-66 was amplified from a human cDNA clone, KIAA0886, from the Kazusa DNA Research Institute and used to generate a construct for expressing AP-66 protein as described by GrandPre et al (2000). The production of Sema3A, co-precipitation and Western analysis were described previously (He & Tessier-Lavigne, 1997).

Growth cone collapse assays. Chick E13 dorsal root ganglion (DRG) and E7 retina were isolated and cultured as described previously (Luo et al., 1993; He and Tessier-Lavigne, 1997; Fournier et al., 2001). Overnight cultured DRG explants were used for growth cone collapse assays. To assess the effects of PI-PLC treatment, cultures were pre-incubated with 2 U/ml PI-PLC for 30 min prior to treatment with individual test proteins for an additional 30 min. To express NgR in E7 retinal ganglion neurons, we infected the explants for 24 hr. Some cultures infected with flag-NgR or LacZ were processed for cytohistochemical staining to verify protein expression. After incubation with each test protein for 30 min, retinal explants were fixed in 4% paraformaldehyde and 15% sucrose, followed by staining with rhodamine-conjugated phalloidin.

Neurite outgrowth assay. P7-9 rat cerebellar neurons were dissected and cultured as described previously (Huang et al., 1999). In brief, 96-well plates were first coated with solubilized nitrocellulose and preincubated with 5 mg/ml poly-D-lysine (Sigma). Purified proteins in a 2-ml drop volume were placed in the center of these wells and incubated for 4 hr at 37° C. Cerebellar neurons were then plated at a density of 1×10⁵ cells per well. The cells were cultured for 24 hr prior to fixation with 4% parafonnaldehyde and staining with an anti-b-tubulin antibody (TuJ, Covance).

Exemplary OMgp Binding Agents. An AP-OMgp fusion protein, prepared as described above, was used to evaluate the OMgp binding affinity of a variety of candidate binding agents. The selected binding assay formats are guided by structural requirements of the candidate agents and include COS-expression, solid phase ELISA-type assay, and fluorescent polarization assays. Candidate agents were selected from natural and synthetic peptide libraries biased to natural NgR LRR (supra) sequences, OMgp-specific monoclonal antibody (Mab) and Mab fragment libraries, a commercial fungal extract library, and a synthetic combinatorial organo-pharmacophore-biased library. Selected exemplary high affinity OMgp-specific binding agents subject to in vivo activity assays (below) are shown in Table 2.

TABLE 2
Selected exemplary high-affinity OMgp-specific binding agents;
(u), structure not yet determined.

| | | | Sequence/ | Binding |
|---|---|---|---|---|
| OMgp Binding Agent | | Class/Source | Structure | Assay |
| 1. | hNR260/308 | natural peptide | SEQ ID NO:1 | + + + + |
| 2. | mNR260/308 | natural peptide | SEQ ID NO:2 | + + + + |
| 3. | rNR260/308 | natural peptide | SEQ ID NO:3 | + + + + |
| 4. | s1NR260/308 | synthetic peptide | SEQ ID NO:4 | + + + + |
| 5. | s2NR260/308 | synthetic peptide | SEQ ID NO:5 | + + + + |
| 6. | s3NR260/308 | synthetic peptide | SEQ ID NO:6 | + + + + |
| 7. | OM-H2276 | monoclonal antibody | IgG | + + + + |
| 8. | OM-H5831 | monoclonal antibody | IgG | + + + + |
| 9. | OMF-H7712 | Fab fragment (Mab) | IgG Fab2 | + + + + |
| 10. | OMF-H6290 | Fab fragment (Mab) | IgG Fab2 | + + + + |
| 11. | XR-178892 | fungal extract library | natural (u) | + + + + |
| 12. | XR-397344 | fungal extract library | natural (u) | + + + + |
| 13. | XR-573632 | fungal extract library | natural (u) | + + + + |
| 14. | SY-73273M | combinatorial library | synthetic (u) | + + + + |
| 15. | SY-32340L | combinatorial library | synthetic (u) | + + + + |
| 16. | SY-95734E | combinatorial library | synthetic (u) | + + + + |

Corticospinal Tract (CST) Regeneration Assay. High affinity OMgp binding agents demonstrating inhibition of OMgp-mediated in vitro axon growth cone collapse as described above are assayed for their ability to improve corticospinal tract (CST) regeneration following thoracic spinal cord injury by promoting CST regeneration into human Schwann cell grafts in the methods of Guest et al. (1997, supra). For these data, the human grafts are placed to span a midthoracic spinal cord transection in the adult nude rat, a xenograft tolerant strain. OMgp binding agents determined to be effective in in vitro collapse assays are incorporated into a fibrin glue and placed in the same region. Anterograde tracing from the motor cortex using the dextran amine tracers, Fluororuby (FR) and biotinylated dextran amine (BDA), are performed. Thirty-five days after grafting, the CST response is evaluated qualitatively by looking for regenerated CST fibers in or beyond grafts and quantitatively by constructing camera lucida composites to determine the sprouting index (SI), the position of the maximum termination density (MTD) rostral to the GFAP-defined host/graft interface, and the longitudinal spread (LS) of bulbous end terminals. The latter two measures provide information about axonal die-back. In control animals (graft only), the CST do not enter the SC graft and undergo axonal die-back. As shown in Table 3, the exemplified binding agents dramatically reduce axonal die-back and cause sprouting and these in vivo data are consistent with the corresponding growth cone collapsing activity.

TABLE 3
In Vitro and Vivo Neuronal Regeneration with Exemplary OMgp
Binding Agents.

| | | Collapse | Reduced | |
|---|---|---|---|---|
| OMgp Binding Agent | | Inhibition | Die-Back | Promote Sprouting |
| 1. | hNR260/308 | + + + + | + + + + | + + + + |
| 2. | mNR260/308 | + + + + | + + + + | + + + + |
| 3. | rNR260/308 | + + + | + + + | + + + |
| 4. | s1NR260/308 | + + + + | + + + + | + + + + |
| 5. | s2NR260/308 | + + + | + + + | + + + |
| 6. | s3NR260/308 | + + + + | + + + + | + + + + |
| 7. | OM-H2276 | + + + + | + + + + | + + + + |
| 8. | OM-H5831 | + + + + | + + + + | + + + + |
| 9. | OMF-H7712 | + + + | + + + | + + + |
| 10. | OMF-H6290 | + + + | + + + | + + + |
| 11. | XR-178892 | + + + | + + + | + + + |
| 12. | XR-397344 | + + + + | + + + + | + + + + |
| 13. | XR-573632 | + + + + | + + + + | + + + + |
| 14. | SY-73273M | + + + | + + + | + + + |
| 15. | SY-32340L | + + + + | + + + + | + + + + |
| 16. | SY-95734E | + + + | + + + | + + + |

Peripheral Nerve Regeneration Assay. High affinity OMgp binding agents demonstrating inhibition of OMgp-mediated in vitro axon growth cone collapse as described above are also incorporated in the implantable devices described in U.S. Pat. No. 5,656,605 and tested for the promotion of in vivo regeneration of peripheral nerves. Prior to surgery, 18 mm surgical-grade silicon rubber tubes (I.D. 1.5 mm) are prepared with or without guiding filaments (four 10-0 monofilament nylon) and filled with test compositions comprising the binding agents of Table 2. Experimental groups consist of: 1. Guiding tubes plus Biomatrix 1™ (Biomedical Technologies, Inc., Stoughton, Mass.); 2. Guiding tubes plus Biomatrix plus filaments; 3-23. Guiding tubes plus Biomatrix 1™ plus binding agents.

The sciatic nerves of rats are sharply transected at mid-thigh and guide tubes containing the test substances with and without guiding filaments sutured over distances of approximately 2 mm to the end of the nerves. In each experiment, the other end of the guide tube is left open. This model simulates a severe nerve injury in which no contact with the distal end of the nerve is present. After four weeks, the distance of regeneration of axons within the guide tube is tested in the surviving animals using a functional pinch test. In this test, the guide tube is pinched with fine forceps to mechanically stimulate sensory axons. Testing is initiated at the distal end of the guide tube and advanced proximally until muscular contractions are noted in the lightly anesthetized animal. The distance from the proximal nerve transection point is the parameter measured. For histological analysis, the guide tube containing the regenerated nerve is preserved with a fixative. Cross sections are prepared at a point approximately 7 mm from the transection site. The diameter of the regenerated nerve and the number of myelinated axons observable at this point are used as parameters for comparison.

Measurements of the distance of nerve regeneration document therapeutic efficacy. Similarly, plots of the diameter of the regenerated nerve measured at a distance of 7 mm into the guide tube as a function of the presence or absence of one or more binding agents demonstrate a similar therapeutic effect of all 16 tested. No detectable nerve growth is measured at the point sampled in the guide tube with the matrix-forming material alone. The presence of guiding filaments plus the matrix-forming material (no agents) induces only very minimal regeneration at the 7 mm measurement point, whereas dramatic results, as assessed by the diameter of the regenerating nerve, are produced by the device which consisted of the guide tube, guiding filaments and binding agent compositions. Finally, treatments using guide tubes comprising either a matrix-forming material alone, or a matrix-forming material in the presence of guiding filaments, result in no measured growth of myelinated axons. In contrast, treatments using a device comprising guide tubes, guiding filaments, and matrix containing binding agents compositions consistently result in axon regeneration, with the measured number of axons being increased markedly by the presence of guiding filaments.

OMgp-Specific Monoclonal Antibodies Promote Axon Regeneration In Vivo. In these experiments, our OM-H2276 and OM-H5831 OMgp-specific monoclonal antibodies are shown to promote axonal regeneration in the rat spinal cord. Tumors producing our OMgp-specific antibodies, implantation protocols and experimental design are substantially as used for IN-1 as described in Schnell et al., Nature Jan. 18, 1990; 343(6255):269-72. In brief, our OM-H2276 and OM-H5831 monoclonal antibodies are applied intracerebrally to young rats by implanting antibody-producing tumours. In 2-6-week-old rats we make complete transections of the corticospinal tract, a major fibre tract of the spinal cord, the axons of which originate in the motor and sensory neocortex. Previous studies have shown a complete absence of cortico-spinal tract regeneration after the first postnatal week in rats, and in adult hamsters and cats. In our treated rats, significant sprouting occurs at the lesion site, and fine axons and fascicles can be observed up to 7-11 mm caudal to the lesion within 2-3 weeks. In control rats, a similar sprouting reaction occurs, but the maximal distance of elongation rarely exceeded 1 mm. These results demonstrate the capacity for CNS axons to regenerate and elongate within differentiated CNS tissue after neutralization of OMgp-mediated axon growth inhibtion.

OMgp-Specific Monoclonal Antibody Fragments Promote Axon Regeneration in Vivo. In these experiments, OMgp-specific monoclonal antibody fragments are shown to promote sprouting of Purkinje cell axons. Experimental protocols were adapted from Buffo et al., 2000, J Neuroscience 20, 2275-2286.

Animals and surgical procedures. Adult Wistar rats (Charles River, Calco, Italy) are deeply anesthetized by means of intraperitoneal administration of a mixture of ketamine (100 mg/kg, Ketalar; Bayer, Leverkusen, Germany) and xylazine (5 mg/kg, Rompun; Bayer).

Fab fragment or antibody injections are performed as previously described (Zagrebelsky et al., 1998). Animals are placed in a stereotaxic apparatus, and the dorsal cerebellar vermis exposed by drilling a small hole on the posterosuperior aspect of the occipital bone. The meninges are left intact except for the small hole produced by the injection pipette penetration. In test rats a recombinant Fab fragment of the OM-H2276 and OM-H5831 antibodies (produced in E. coli), which neutralizes OMgp-associated axon growth cone collapse in vitro is injected into the cerebellar parenchyma. Three 1 μl injections of Fab fragments in saline solution (5 mg/ml) are performed 0.5-1 mm deep along the cerebellar midline into the dorsal vermis (lobules V-VII). The injections are made by means of a glass micropipette connected to a PV800 Pneumatic Picopump (WPI, New Haven, Conn.). The frequency and duration of pressure pulses are adjusted to inject 1 μl of the solution during a period of ˜10 min. The pipette is left in situ for 5 additional minutes to avoid an excessive leakage of the injected solution. As a control, an affinity-purified F(ab′)₂ fragment of a mouse anti-human IgG (Jackson ImmunoResearch Laboratories, West Grove, Pa.) is applied to another set of control rats using the same procedure. Survival times for these two experimental sets are 2, 5, 7 and 30 d (four animals for each time point). An additional set of intact animals are examined as untreated controls.

Histological procedures. At different survival times after surgery, under deep general anesthesia (as above), the rats are transcardially perfused with 1 ml of 4% paraformaldehyde in 0.12 M phosphate buffer, pH 7.2-7.4. The brains are immediately dissected, stored overnight in the same fixative at 4° C., and finally transferred in 30% sucrose in 0.12 M phosphate buffer at 4° C. until they sink. The cerebella are cut using a freezing microtome in several series of 30-μm-thick sagittal sections. One series is processed for NADPH diaphorase histochemistry. These sections are incubated for 3-4 hr in darkness at 37° C. in a solution composed of -NADPH (1 mg/ml, Sigma, St. Louis, Mo.) and nitroblue tetrazolium (0.2 mg/ml, Sigma) in 0.12 M phosphate buffer with 0.25% Triton X-100. In some cases (two animals per treated and control sets at 2 and 5 d survival), microglia are stained by incubating one section series with biotinylated Griffonia simplicifolia isolectin B4 [1:100 in phosphate buffer with 0.25% Triton X-100; Sigma (Rossi et al., 1994a)] overnight at 4° C. Sections are subsequently incubated for 30 min in the avidin-biotin-peroxidase complex (Vectastain, ABC Elite kit, Vector, Burlingame, Calif.) and revealed using the 3,3′ diaminobenzidine (0.03% in Tris HCl) as a chromogen.

All of the other series are first incubated in 0.3% H₂O₂ in PBS to quench endogenous peroxidase. Then, they are incubated for 30 min at room temperature and overnight at 4° C. with different primary antibodies: anti-calbindin D-28K (monoclonal, 1:5000, Swant, Bellinzona, Switzerland), to visualize Purkinje cells; anti-c-Jun (polyclonal, 1:1000, Santa Cruz Biotechnology, Santa Cruz, Calif.); and anti-CD11b/c (monoclonal OX-42, 1:2000, Cedarlane Laboratories, Hornby, Ontario) to stain microglia. All of the antibodies are diluted in PBS with 0.25% Triton X-100 added with either normal horse serum or normal goat serum depending on the species of the second antibody. Immunohistochemical staining is performed according to the avidin-biotin-peroxidase method (Vectastain, ABC Elite kit, Vector) and revealed using the 3,3′ diaminobenzidine (0.03% in Tris HCl) as a chromogen. The reacted sections are mounted on chrome-alum gelatinized slides, air-dried, dehydrated, and coverslipped.

Quantitative analysis. Quantification of reactive Purkinje cells in the different experiments is made by estimating the neurons labeled by c-Jun antibodies as previously described (Zagrebelsky et al., 1998). For each animal, three immunolabeled sections are chosen. Only vermal sections close to the cerebellar midline that contain the injection sites are considered. The outline of the selected sections is reproduced using the Neurolucida software (MicroBrightField, Colchester, Vt.) connected to an E-800 Nikon microscope, and the position of every single-labeled cell carefully marked. The number of labeled cells present in the three reproduced sections is averaged to calculate values for every individual animal, which are used for statistical analysis carried out by Student's t test.

A morphometric analysis of Purkinje axons in the different experimental conditions for each animal, is performed using three anti-calbindin-immunolabeled sections, contiguous to those examined for c-Jun, as described in Buffo et al. (supra). Morphometric measurements are made on 200×250 μm areas of the granular layer chosen by superimposing a grid of this size on the section. The selected areas encompass most of the granular layer depth and contain only minimal portions of Purkinje cell layer or axial white matter. In each of the selected sections is sampled one area from the dorsal cortical lobules and one from the ventral cortical lobules. In addition, to sample from the different parts of these two cortical regions, areas from different lobules are selected in the three sections belonging to each individual animal, one area in each of lobules V, VI, and VII and one in lobules I, II, and IX. All of the anti-calbindin-immunolabeled Purkinje axon segments contained within the selected areas are reproduced using the Neurolucida software (MicroBrightField) connected to an E-800 Nikon microscope with 20× objective, corresponding to 750× magnification on the computer screen. Each labeled axon segment or branch is reproduced as a single profile. From these reproductions the software calculates the number of axon profiles, their individual length, and the total length of all the reproduced segments, the mean profile length (total length/number of profiles), and the number of times that the axons crossed a 25×25 μm grid superimposed on the selected area. Data calculated from the different areas in the three sections sampled from each cerebellum are averaged to obtain values for every individual animal. Statistical analysis is performed on the latter values (n=4 for all groups at all time points) by Student's t test and paired t test.

Our results reveal significant promotion of sprouting of Purkinje cell axons in test rats subject to our OM-H2276 and OM-H5831 OMgp-specific monoclonal antibody fragments as compared with the control animals.

### CITED REFERENCES

Arquint, et al. (1987). Molecular cloning and primary structure of myelin-associated glycoprotein. Proc Natl Acad Sci USA 84, 600-604.

Bhat, S., Silberberg, D. H.(1986). Oligodendrocyte cell adhesion molecules are related to neural cell adhesion molecule (N-CAM). J Neurosci 6, 3348-3354.

Bartsch, U., et al. (1995). Lack of evidence that myelin-associated glycoprotein is a major inhibitor of axonal regeneration in the CNS. Neuron 15, 1375-1381.

Bregman, et al. (1995). Recovery from spinal cord injury mediated by antibodies to neurite growth inhibitors. Nature 378, 498-501.

Cammer, et al. (1985). Immunocytochemical localization of 5′-nucleotidase in oligodendroglia and myelinated fibers in the central nervous system of adult and young rats. Brain Res 352, 89-96.

Caroni, P. and Schwab, M. E. (1988a). Antibody against myelin-associated inhibitor of neurite growth neutralizes nonpermissive substrateproperties of CNS white matter. Neuron 1, 85-96.

Caroni, P. and Schwab, M. E.. (1988b). Two membrane protein fractions from rat central myelin with inhibitory properties for neurite growth and fibroblast spreading. J Cell Biol 106, 1281-1288.

Chen, et al. (2000). Nogo-A is a myelin-associated neurite outgrowth inhibitor and an antigen for monoclonal antibody IN-1. Nature 403, 434-439.

David, S., and Aguayo, A. J. (1981). Axonal regeneration into peripheral nervous system “bridges” after central nervous system injury in adult rats. Science 214, 931-933.

Davies, et al. (1997). Regeneration of adult axons in white matter tracts of the central nervous system. Nature 390, 680-683.

Flanagan, J. G., Leder, P. (1990). The kit ligand: a cell surface molecule altered in steel mutant fibroblasts. Cell 63, 185-194.

Flanagan, J. G. and Cheng, H. J. (2000). Alkaline phosphatase fusion proteins for molecular characterization and cloning of receptors and their ligands. Methods Enzymol 327, 198-210.

Fournier, A. E., Strittmatter, S. M.(2001). Repulsive factors and axon regeneration in the CNS. Curr Opin Neurobiol 11, 89-94.

Fournier, et al. (2001). Identification of a receptor mediating Nogo-66 inhibition of axonal regeneration. Nature 409, 341-346.

GrandPre, et al. (2000). Identification of the Nogo inhibitor of axon regeneration as a Reticulon protein. Nature 403, 439-444.

Habib et al. (1998a) Expression of the oligodendrocyte-myelin glycoprotein by neurons in the mouse central nervous system. J Neurochem 70, 1704-1711.

Habib et al. (1998b) The OMgp gene, a second growth suppressor within the NF1 gene.

He, Z., Tessier-Lavigne, M. (1997). Neuropilin is a receptor for the axonal chemorepellent Semaphorin III. Cell 90, 739-751.

Horner, P. J,. Gage, F. H. (2000). Regenerating the damaged central nervous system. Nature 407, 963-970.

Huang, et al. (1999). A therapeutic vaccine approach to stimulate axon regeneration in the adult mammalian spinal cord. Neuron 24, 639-647.

Kobe, B., Deisenhofer, J. (1995). Proteins with leucine-rich repeats. Curr Opin Struct Biol 5, 409-416.

Koch, et al. (1997). Expression of the immunoglobulin superfamily cell adhesion molecule F3 by oligodendrocyte-lineage cells. Glia 19, 199-212.

Kuhn et al. (1999). Myelin and collapsin-1 induce motor neuron growth cone collapse through different pathways: inhibition of collapse by opposing mutants of rac1. J Neurosci. 19, 1965-1975.

Lehmann et al. (1999). Inactivation of Rho signaling pathway promotes CNS axon regeneration. J Neurosci 19, 7537-7547.

Li, M., et al. (1996). Myelin-associated glycoprotein inhibits neurite/axon growth and causes growth cone collapse. J Neurosci Res 46, 404-414.

Low, M. G. (1987). Biochemistry of the glycosyl-phosphatidylinositol membrane protein anchors. Biochem J 244, 1-13.

Luo, Y., Raible, D., Raper, J. A. (1993). Collapsin: a protein in brain that induces the collapse and paralysis of neuronal growth cones. Cell 75, 217-227.

McKerracher, et al. (1994). Identification of myelin-associated glycoprotein as a major myelin-derived inhibitor of neurite growth. Neuron 13, 805-811.

McKeon, et al. (1991). Reduction of neurite outgrowth in a model of glial scarring following CNS injury is correlated with the expression of inhibitory molecules on reactive astrocytes. J Neurosci 11, 3398-3411.

Mikol, D. D., Stefansson, K. (1988). A phosphatidylinositol-linked peanut agglutinin-binding glycoprotein in central nervous system myelin and on oligodendrocytes. J Cell Biol 106, 1273-1279.

Mikol, D. D., Gulcher, J. R., Stefansson, K. (1990). The oligodendrocyte-myelin glycoprotein belongs to a distinct family of proteins and contains the HNK-1 carbohydrate. J Cell Biol 110, 471-479.

Moon, et al. (2001). Regeneration of CNS axons back to their target following treatment of adult rat brain with chondroitinase ABC. Nat Neurosci 4, 465-466.

Mukhopadhyay, et al.. (1994). A novel role for myelin-associated glycoprotein as an inhibitor of axonal regeneration. Neuron 13, 757-767.

Nakashiba, et al. (2000). Netrin-G1: a novel glycosyl phosphatidylinositol-linked mammalian netrin that is functionally divergent from classical netrins. J Neurosci 20, 6540-6550.

Neve et al. (1997) Introduction of the glutamate receptor subunit 1 into motor neurons in vitro and in vivo using recombinant herpes simplex virus. Neuroscience 79:435-447

Niederost, et al. (1999). Bovine CNS myelin contains neurite growth-inhibitory activity associated with chondroitin sulfate proteoglycans. J Neurosci 19, 8979-8989.

Norton, W. T., and Poduslo, S. E. (1973). Myelination in rat brain: method of myelin isolation. J. Neurochem. 21, 749-757.

O'Leary, D. D., Wilkinson, D. G.(1999). Eph receptors and ephrins in neural development. Curr Opin Neurobiol 9, 65-73.

Prinjha, et al. (2000). Inhibitor of neurite outgrowth in humans. Nature 403, 383-384.

Ranscht, B., Dours-Zimmermann, M. T. (1991). T-cadherin, a novel cadherin cell adhesion molecule in the nervous system lacks the conserved cytoplasmic region. Neuron 7, 391-402.

Raper, J. A. (2000). Semaphorins and their receptors in vertebrates and invertebrates. Curr Opin Neurobiol 10, 88-94.

Ren, et al. (1999). Regulation of the small GTP-binding protein Rho by cell adhesion and the cytoskeleton. EMBO J. 18, 578-585.

Salzer, et al. (1987). The amino acid sequences of the myelin-associated glycoproteins: homology to the immunoglobulin gene superfamily. J Cell Biol 104, 957-965.

Savio, T. and Schwab, M. E.. (1989). Rat CNS white matter, but not gray matter, is nonpermissive for neuronal cell adhesion and fiber outgrowth. J Neurosci 9, 1126-1133.

Schnell, L., and Schwab, M. E. (1990). Axonal regeneration in the rat spinal cord produced by an antibody against myelin-associated neurite growth inhibitors. Nature 343, 269-272.

Schafer, et al. (1996). Disruption of the gene for the myelin-associated glycoprotein improves axonal regrowth along myelin in C57BL/Wlds mice. Neuron 16, 1107-1113.

Schwab, M. E. and Caroni, P. (1988). Oligodendrocytes and CNS myelin are nonpermissive substrates for neurite growth and fibroblast spreading in vitro. J Neurosci 2381-2393.

Schwab, M. E., and Bartholdi, D. (1996). Degeneration and regeneration of axons in the lesioned spinal cord. Physiol. Rev. 76, 319-370.

Seidenbecher, et al. (1995). Brevican, a chondroitin sulfate proteoglycan of rat brain, occurs as secreted and cell surface glycosylphosphatidylinositol-anchored isoforms. J Biol Chem 270, 27206-27212.

Song, H. and Poo, M. (2001). The cell biology of neuronal navigation. Nat Cell Biol 3, E81-8

Spillmann, et al. (1998). Identification and characterization of a bovine neurite growth inhibitor (bNI-220). J Biol Chem 273, 19283-19293.

Tang, et al. (1997). Soluble Myelin-Associated Glycoprotein (MAG) Found in Vivo Inhibits Axonal Regeneration. Mol Cell Neurosci 9, 333-346.

Tessier-Lavigne, M. and Goodman, C. S. (1996) The molecular biology of axon guidance. Science, 274:1123-1133.

Tessier-Lavigne, M, Goodman, C. S.(2000). Perspectives: neurobiology. Regeneration in the Nogo zone. Science 287, 813-814.

Vinson et al. (2001). Myelin-associated glycoprotein interacts with ganglioside GT1b. A mechanism for neurite outgrowth inhibition. J. Biol. Chem. 276, 20280-20285.

Xu, et al. (1998). Human semaphorin K1 is glycosylphosphatidylinositol-linked and defines a new subfamily of viral-related semaphorins. J Biol Chem 273, 22428-22434.

Zimmermann, H. (1992). 5′-Nucleotidase: molecular structure and functional aspects. Biochem J 285, 345-65.

The foregoing descriptions of particular embodiments and examples are offered by way of illustration and not by way of limitation. All publications and patent applications cited in this specification and all references cited therein are herein incorporated by reference as if each individual publication or patent application or reference were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for reducing axon growth inhibition mediated by oligodendrocyte-myelin glycoprotein (OMgp) and detecting resultant reduced axon growth inhibition, the method comprising steps:
contacting a mixture comprising an axon and isolated OMgp with an agent and under conditions wherein but for the presence of the agent, the axon is subject to growth inhibition mediated by the OMgp; and
detecting resultant reduced axon growth inhibition.

2. A method according to claim 1, wherein the isolated OMgp consists essentially of OMgp.

3. A method according to claim 1, wherein the isolated OMgp consists essentially of OMgp, wherein the OMgp is soluble and GPI-cleaved.

4. A method according to claim 1, wherein the OMgp is recombinantly expressed on a surface of a cell.

5. A method according to claim 1, wherein the mixture is in vitro.

6. A method according to claim 1, wherein the agent is a candidate agent not previously characterized to bind OMgp nor reduce axon growth inhibition mediated by OMgp and the detecting step characterizes the candidate agent as reducing axon growth inhibition mediated by OMgp.

7. A method according to claim 1, wherein the agent is a candidate agent not previously characterized to reduce axon growth inhibition mediated by OMgp and the detecting step characterizes the candidate agent as reducing axon growth inhibition mediated by OMgp.

8. A method according to claim 1, wherein the agent comprises an OMgp-specific antibody fragment.

9. A method according to claim 1, wherein the agent is soluble Nogo receptor (NgR) peptide sufficient to specifically bind the OMgp and competitively inhibit binding of the OMgp to NgR

10. A method for reducing axon growth inhibition mediated by OMgp and detecting resultant reduced axon growth inhibition, the method comprising steps:
contacting a mixture comprising an axon and OMgp with an exogenous OMgp-specific binding agent and under conditions wherein the agent binds the OMgp and but for the presence of the agent, the axon is subject to growth inhibition mediated by the OMgp, and
detecting resultant reduced axon growth inhibition.

11. A method according to claim 10, wherein the mixture is in vitro.

12. A method according to claim 10, wherein the agent is a candidate agent not previously characterized to bind OMgp nor reduce axon growth inhibition mediated by OMgp and the detecting step characterizes the candidate agent as reducing axon growth inhibition mediated by OMgp.

13. A method according to claim 10, wherein the agent is a candidate agent not previously characterized to reduce axon growth inhibition mediated by OMgp and the detecting step characterizes the candidate agent as reducing axon growth inhibition mediated by OMgp.

14. A method according to claim 10, wherein the agent comprises an OMgp-specific antibody fragment.

15. A method according to claim 10, wherein the agent is soluble NgR peptide sufficient to specifically bind the OMgp and competitively inhibit binding of the OMgp to NgR.

16. A method according to claim 10, wherein the agent is soluble NgR peptide sufficient to specifically bind the OMgp and competitively inhibit binding of the OMgp to NgR, wherein the peptide consists essentially of a sequence within SEQ ID NO:1 at least six residues in length.

17. A method for characterizing an agent as inhibiting binding of NgR to OMgp, the method comprising the steps of:
incubating a mixture comprising NgR, OMgp and an agent under conditions whereby but for the presence of the agent, the NgR and OMgp exhibit a control binding; and
detecting a reduced binding of the NgR to the OMgp, indicating that the agent inhibits binding of the NgR to the OMgp.

18. A method according to claim 17, wherein at least one of the NgR and OMgp is soluble and GPI-cleaved.

19. A method according to claim 17, wherein one of the NgR and OMgp is soluble and GPI-cleaved and the other is membrane-bound.

20. A method according to claim 17, wherein at least one of the NgR and OMgp is recombinantly expressed on a surface of a cell.
